(19) United States
(12) Patent Application Publication
(10) Pub. No.: US 2014/0254765 A1
(43) Pub. Date: Sep. 11, 2014

(54) DISPLAY CONTROL APPARATUS, DISPLAY CONTROL METHOD, AND COMPUTER-READABLE STORAGE MEDIUM STORING PROGRAM

(71) Applicant: CANON KABUSHIKI KAISHA, Tokyo (JP)

(72) Inventor: Hiroki Asai, Kawasaki-shi (JP)

(73) Assignee: CANON KABUSHIKI KAISHA, Tokyo (JP)

(21) Appl. No.: 14/198,352

(22) Filed: Mar. 5, 2014

(30) Foreign Application Priority Data
Mar. 6, 2013 (JP) 2013-044542

(51) Int. Cl. A61B 6/00

(52) U.S. Cl. CPC A61B 6/461
USPC 378/98.5; 378/98.2

(57) ABSTRACT

A display control apparatus includes a display control unit configured to display first image data and second image data in different display modes on a display unit. The first image data is generated by detecting, with use of a radiation detection unit, radiation that has transmitted through a subject, and the second image data is generated by subjecting the first image data to predetermined image processing.

### BACKGROUND OF THE INVENTION

1. Field of the Invention

The present invention relates to a technique for displaying image data generated by detecting radiation that has transmitted through a subject.

2. Description of the Related Art

In recent years, there has been put to practical use a radiation imaging system which generates radiation image data by directly digitizing a radiation image using a flat panel detector (FPD), which is a radiation sensor formed by adhering a scintillator to a solid-state image sensor used for large screens. The radiation imaging system employing such digitizing scheme is widely used in place of a conventional radiation imaging system employing an analog method.

The radiation imaging system employing the digital method has reduced the time taken to check the data of a captured radiation image. On the other hand, since resolution of image data has been increased, the time required from capturing a radiation image to displaying the image data tends to increase. In other words, a delay in displaying the image data is likely to occur. As a result, the determination of the imaging state (e.g., whether the exposure is appropriate or whether the image of the body part is captured at an appropriate angle) may be delayed. Thus, if an imaging failure occurs, the workflow of X-ray examination may be disturbed. If a result of the imaging can be checked at an early stage, whether a retake of an image (reshooting) is necessary can be promptly determined and the workflow of the X-ray examination can be favorably maintained.

From this viewpoint, Japanese Patent Application Laid-Open No. 2009-45430 discusses a technique in which a radiation image system promptly displays a preview of compressed image data obtained by reducing the information amount of radiation image data. Further, Japanese Patent Application Laid-Open No. 2006-26083 discusses a technique for promptly displaying a preview of the data of a captured radiation image before completion of an offset correction to the data.

However, although such preview images can be satisfactorily used to determine whether reshooting is necessary, they may not be appropriate to be used for diagnosis. This is because, for example, the information amount of the preview images is reduced or the preview images are not yet subjected to appropriate correction processing.

### SUMMARY OF THE INVENTION

According to an aspect of the present invention, a display control apparatus includes a display control unit configured to control a display unit to display first image data and second image data in different display modes on the display unit, the first image data being generated by detecting, with use of a radiation detection unit, radiation that has transmitted through a subject, and the second image data being generated by subjecting the first image data to predetermined image processing.

Further features of the present invention will become apparent from the following description of exemplary embodiments with reference to the attached drawings.

### BRIEF DESCRIPTION OF THE DRAWINGS

FIG. 1 is a schematic diagram illustrating an example of a schematic configuration of a radiation imaging system according to a first exemplary embodiment of the present invention.

FIG. 2 is a block diagram illustrating a configuration of each apparatus of the radiation imaging system according to the first exemplary embodiment of the present invention.

FIG. 3 is a flowchart illustrating an operation of a radiation imaging apparatus according to the first exemplary embodiment.

FIG. 4 illustrates an example of a progress bar displayed together with first image data.

FIG. 5 illustrates a display example of the first image data.

FIG. 6 illustrates an example of a display screen of a display unit according to a second exemplary embodiment of the present invention.

FIG. 7 is a detailed configuration of a display management unit according to the first exemplary embodiment.

FIG. 8 is a flowchart illustrating an operation of a control personal computer (PC) according to the first exemplary embodiment.

FIG. 9 is a schematic diagram illustrating another example of the schematic configuration of the radiation imaging system according to the first exemplary embodiment of the present invention.

FIG. 10 illustrates a detailed configuration of a display management unit according to the second exemplary embodiment.

FIG. 11 illustrates another example of the progress bar displayed together with the first image data.

FIG. 12 is a block diagram illustrating an example of a configuration of a radiation imaging apparatus according to an exemplary embodiment of the present invention.

### DESCRIPTION OF THE EMBODIMENTS

Various exemplary embodiments, features, and aspects of the invention will be described in detail below with reference to the drawings.

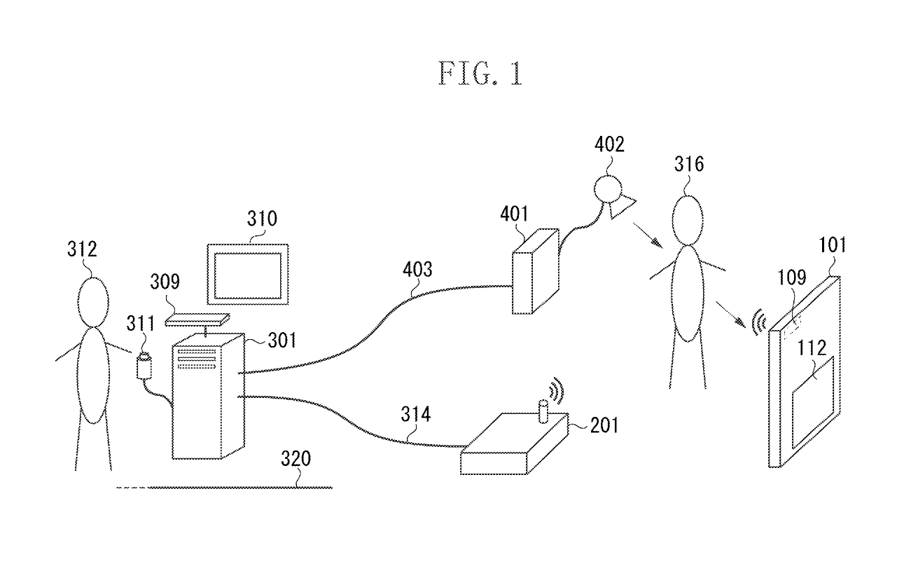

First, a first exemplary embodiment of the present invention will be described. FIG. 1 is a schematic diagram illustrating an example of a configuration of a radiation imaging system according to the first exemplary embodiment of the present invention. A radiation imaging apparatus 101 generates radiation image data in response to radiation. A power supply control circuit 112 serves as a battery for supplying power to the radiation imaging apparatus 101. A wireless communication circuit 109 is used for performing wireless local area network (LAN) communication. A control PC 301 controls the radiation imaging apparatus 101 and a radiation generation apparatus 402 and also performs image processing. An access point 201 performs wireless communication with the radiation imaging apparatus 101. A connection cable 314 is used for wired connection between the access point 201 and the control PC 301. A backbone network 320 is, for example, an in-hospital LAN to which the control PC 301 is connected. An operation panel 309 is an operation panel for the control PC 301. A display 310 displays the data of a captured radiation image, the radiation image data subjected to image processing, and a graphical user interface (GUI). FIG. 1 further illustrates a radiation irradiation switch 311, an operator 312, a patient 316, a radiation control apparatus 401, the radiation generation apparatus 402, and a connection cable 403. The connection cable 403 is used for wired connection between the control PC 301 and the radiation control apparatus 401. The radiation imaging apparatus 101 is an example of a radiation imaging unit.

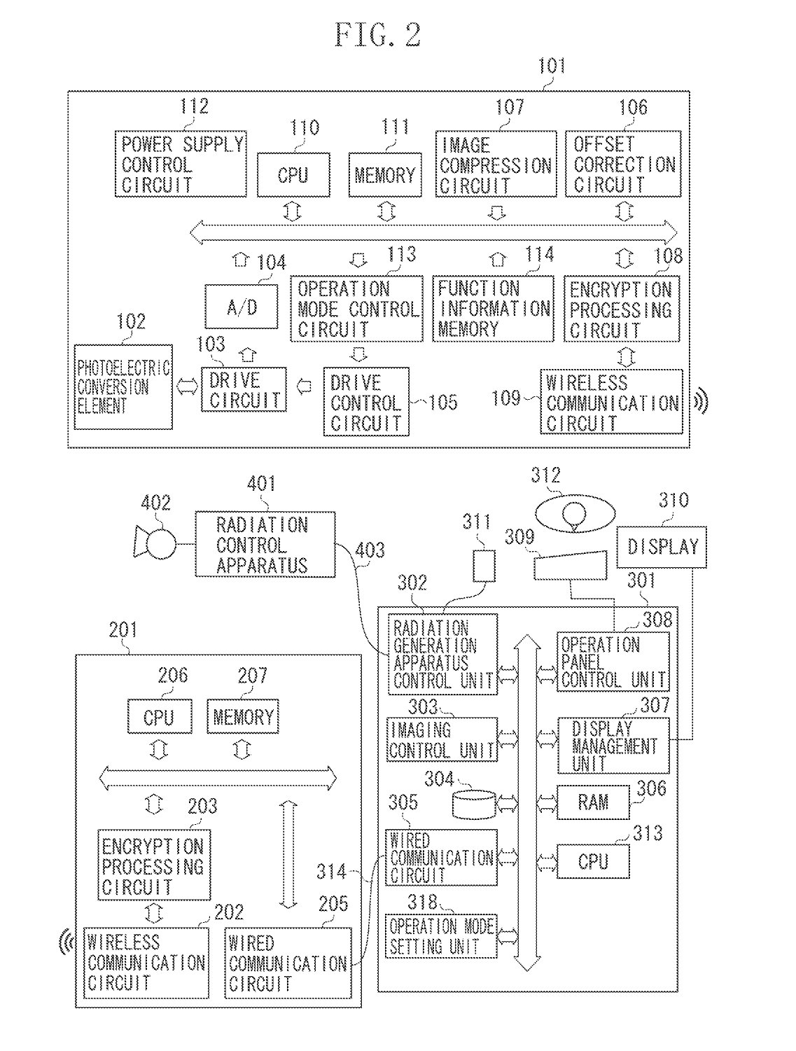

Next, a detailed configuration of each apparatus of the radiation imaging system will be described with reference to a block diagram illustrated in FIG. 2.

The radiation imaging system includes the radiation imaging apparatus 101, the radiation control apparatus 401, the radiation generation apparatus 402, the access point 201, the control PC 301, the operation panel 309, the display 310, and the radiation irradiation switch 311. The display 310 is an example of a display unit.

The radiation imaging apparatus 101 causes an image sensor to capture an image of radiation (e.g., X-ray) that has transmitted through a subject to acquire X-ray image data of the subject.

The radiation imaging apparatus 101 includes a central processing unit (CPU) 110, a memory 111, a photoelectric conversion element 102, a drive circuit 103, an analog-to-digital (A/D) conversion circuit 104, a drive control circuit 105, an operation mode control circuit 113, a function information memory 114, an encryption processing circuit 108, the wireless communication circuit 109, and the power supply control circuit 112.

The CPU 110 controls the entire part of the radiation imaging apparatus 101 by using a program or various types of data stored in the memory 111. The memory 111 stores, for example, a program and various types of data to be used by the CPU 110 for executing various types of processing. Further, the memory 111 stores various types of data acquired by the processing of the CPU 110 and data of captured images. The photoelectric conversion element 102 includes a plurality of pixels arranged in a two-dimensional manner. The main material of the photoelectric conversion element 102 is amorphous silicon. The photoelectric conversion element 102 receives the radiation converted into a visible light and detects it as a radiation image signal.

The drive circuit 103 drives the photoelectric conversion element 102. More precisely, the drive circuit 103 drives the photoelectric conversion element 102 to perform processing for reading out a radiation image signal. The photoelectric conversion element 102 is controlled by the drive circuit 103 to be in either a charge accumulation state or a charge non-accumulation state. The charge non-accumulation state is, for example, a sleep state in which no voltage is applied to the photoelectric conversion element 102, a sensor standby state in which a voltage is applied to the photoelectric conversion element 102, and a sensor readout state in which a radiation image signal is read out by driving the photoelectric conversion element 102.

The A/D conversion circuit 104 converts a radiation image signal, which is an analog signal read out from the drive circuit 103, into a digital radiation image signal. The digital radiation image signal is stored in the memory 111 as radiation image data. The drive control circuit 105 controls the drive circuit 103 based on an instruction from the operation mode control circuit 113.

The operation mode control circuit 113 controls the drive control circuit 105 according to an operation mode designated by a command issued from the control PC 301. The operation mode is, for example, a synchronous radiation mode and an automatic radiation detection mode.

The synchronous radiation mode is the mode in which the radiation imaging apparatus 101 shifts the state of the photoelectric conversion element 102 to the charge accumulation state in response to a press of the radiation irradiation switch 311, and communicates with the radiation generation apparatus 402 to synchronize the radiation emission timing with the charge accumulation period so that radiation is emitted during the period. In the synchronous radiation mode, the radiation generation apparatus 402 notifies the radiation imaging apparatus 101 of the press of the radiation irradiation switch 311 and the completion timing of radiation emission, and the radiation imaging apparatus 101 notifies the radiation generation apparatus 402 of the timing of when the photoelectric conversion element 102 has been shifted to the charge accumulation state, so that the radiation emission timing is synchronized with the charge accumulation period.

The automatic radiation detection mode is the mode in which the radiation imaging apparatus 101 shifts the state of the photoelectric conversion element 102 to the charge accumulation state in accordance with the radiation emission timing so that the radiation generation apparatus 402 can emit the radiation at an arbitrary timing. The function information memory 114 stores information about the functions (operation modes) supported by the radiation imaging apparatus 101. The above-described memory 111 can be used in place of the function information memory 114.

An offset correction circuit 106 performs an offset correction to the radiation image data stored in the memory 111 to remove a dark noise component (dark current data) caused by the radiation imaging apparatus 101 from the radiation image data so that low-noise radiation image data is obtained.

An image compression circuit 107 performs compression processing on the radiation image data stored in the memory 111 so that the generated radiation image data can be transferred to the control PC 301 at a high speed. The compression processing includes data thinning processing and encoding to the Joint Photographic Experts Group (JPEG) format.

According to the present exemplary embodiment, although the offset correction and the image compression are performed by the radiation imaging apparatus 101 using the image processing circuits (the offset correction circuit 106 and the image compression circuit 107), image processing such as basic correction processing (e.g., gain correction and defective pixel correction) and image quality adjustment (e.g., gradation correction) requested by the user may also be performed by the radiation imaging apparatus 101.

The encryption processing circuit 108 encrypts the radiation image data and communication data such as the current processing status of the radiation imaging apparatus 101, and outputs the encrypted data to the wireless communication circuit 109 so that the data is transmitted. If the wireless communication circuit 109 receives encrypted communication data, the encryption processing circuit 108 decodes the encrypted communication data.

The wireless communication circuit 109 transmits the encrypted communication data input from the encryption processing circuit 108, and also outputs the received communication data to the encryption processing circuit 108. The power supply control circuit 112, which includes a battery and a direct-current-to-direct-current (DC-to-DC) converter, supplies power to each circuit. The access point 201 includes a wireless communication circuit 202, an encryption processing circuit 203, a wired communication circuit 205, a CPU 206, and a memory 207.

The CPU 206 controls the entire part of the access point 201 by using a program and various types of data stored in the memory 207. The memory 207 stores, for example, a program and various types of data to be used by the CPU 206 for executing various types of processing. The encryption processing circuit 203 encrypts the communication data and outputs the encrypted data to the wireless communication circuit 202 so that the data is transmitted. When the wireless communication circuit 202 receives encrypted communication data, the encryption processing circuit 203 decodes the encrypted communication data.

The wireless communication circuit 202 transmits the encrypted communication data input from the encryption processing circuit 203, and also outputs the received communication data to the encryption processing circuit 203. The wired communication circuit 205 controls the communication of various types of data and information between the access point 201 and the control PC 301.

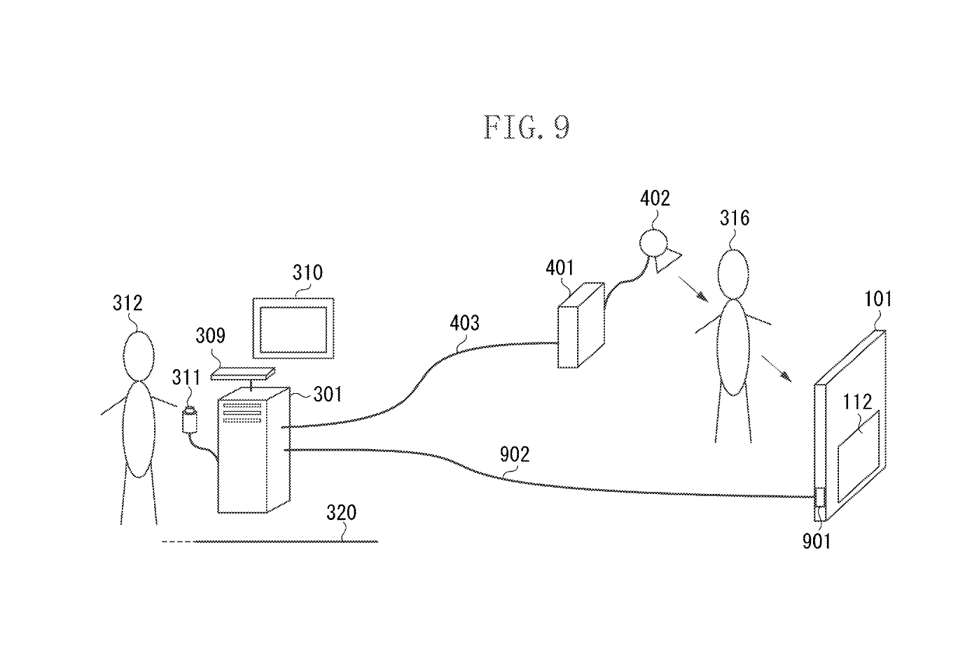

In the description above, although a captured image is transmitted by wireless communication via the encryption processing circuit 108, the wireless communication circuit 109, and the access point 201, the captured image may also be transmitted by wired communication as illustrated in FIG. 9. In FIG. 9, the radiation imaging apparatus 101 includes a connector 901 for wired communication to connect to the control PC 301 using a communication cable 902.

The control PC 301 includes a radiation generation apparatus control unit 302, an imaging control unit 303, an external storage unit 304, a wired communication circuit 305, a CPU 313, a random access memory (RAM) 306, a display management unit 307, an operation panel control unit 308, and an operation mode setting unit 318.

The radiation generation apparatus control unit 302 performs the control associated with the radiation generation performed by the radiation generation apparatus 402, based on an imaging instruction issued by the operator 312. Since the radiation generation apparatus control unit 302 does not have a synchronization control function regarding the timing of radiation emission, the radiation generation apparatus control unit 302 starts emitting radiation when the radiation irradiation switch 311 is pressed, regardless of the state of the radiation imaging apparatus 101.

The imaging control unit 303 controls the radiation imaging performed by the radiation imaging apparatus 101 based on an imaging instruction issued by the operator 312 and an operation mode set by the operation mode setting unit 318.

The external storage unit 304 includes, for example, a hard disk. The external storage unit 304 stores various programs, various types of data, or various types of information. The wired communication circuit 305 controls the communication of various types of data and information between the control PC 301 and the access point 201. The communication cable 314 connects the access point 201 and the control PC 301 in a communicable manner. The CPU 313 controls the entire part of the control PC 301 using a program and various types of data stored in the RAM 306. The RAM 306 temporarily stores the various types of data and various types of information necessary for the processing of the control PC 301.

The display management unit 307 performs various types of control associated with the display of the display 310. The operation panel control unit 308 performs various types of control associated with the operation panel 309. For example, the operation panel control unit 308 switches the display on the operation panel 309 according to an operation of the operation panel 309 performed by the operator 312.

The operation panel 309 is operated by the operator 312. An instruction input by the operator 312 is input to the control PC 301 via the operation panel 309. The radiation irradiation switch 311 is also operated by the operator 312. When the operator 312 presses the radiation irradiation switch 311, an imaging instruction is input to the radiation generation apparatus control unit 302 and the imaging control unit 303 to start the radiation imaging. The display 310 displays various types of images and information based on the control by the display management unit 307. The display management unit 307 displays the processing status of the radiation imaging apparatus 101 and the control PC 301 by using, for example, a progress bar (progress display).

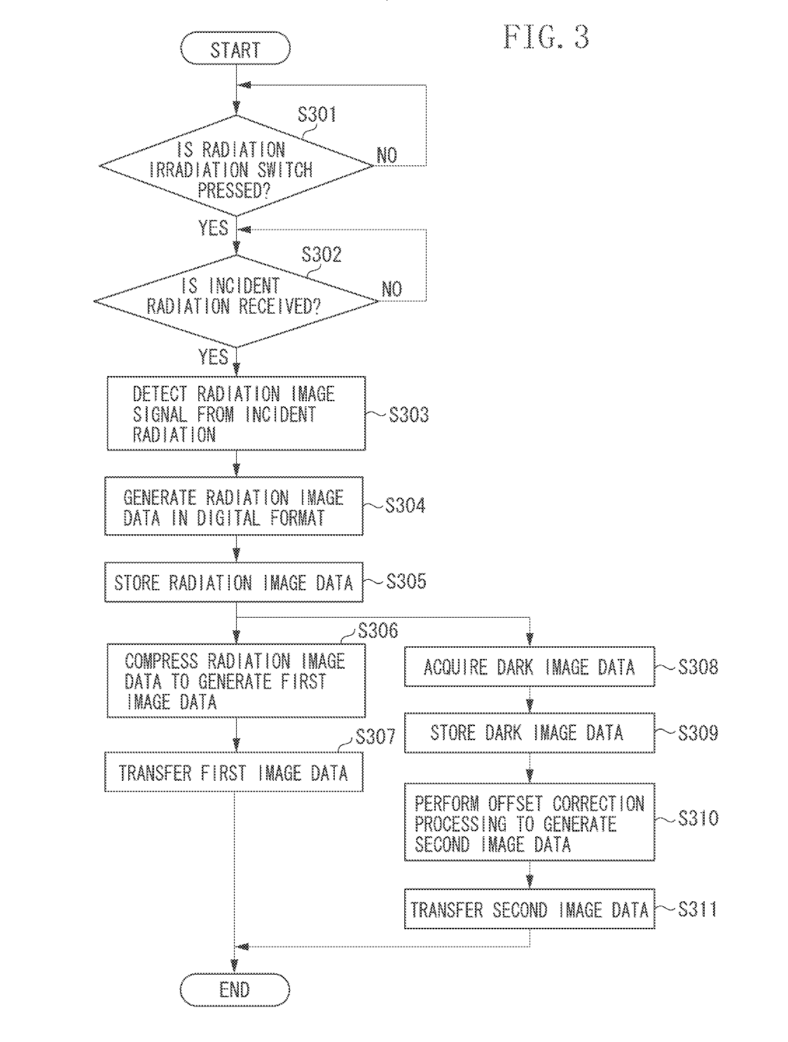

Radiation image data acquisition processing performed by the radiation imaging apparatus 101 in the synchronous radiation mode according to the present exemplary embodiment will be described with reference to a flowchart of an operation by the radiation imaging apparatus 101 illustrated in FIG. 3. First, when the operator 312 presses the radiation irradiation switch 311, radiation is emitted from the radiation generation apparatus 402 to the patient 316. Then, the emitted radiation transmits through the patient 316 and is incident on the radiation imaging apparatus 101.

In step S301, when the radiation imaging apparatus 101 has received a signal indicating that the radiation irradiation switch 311 has been pressed, via the wired communication circuit 305, the access point 201, and the wireless communication circuit 109 (YES in step S301), the processing proceeds to step S302. In step S302, the radiation imaging apparatus 101 changes the state of the photoelectric conversion element 102 to the charge accumulation state to stand by to receive incident radiation. When incident radiation is received (YES in step S302), the processing proceeds to step S303. In step S303, the radiation imaging apparatus 101 converts the incident radiation into a visible light. Then, based on an instruction from the operation mode control circuit 113, the radiation imaging apparatus 101 drives the photoelectric conversion element 102 by using the drive control circuit 105 and the drive circuit 103 to detect the visible light as a radiation image signal. In step S304, the radiation imaging apparatus 101 reads out the detected radiation image signal and converts the radiation image signal, which is an analog signal, into a digital signal by using the A/D conversion circuit 104 to generate radiation image data in a digital format. In step S305, the radiation imaging apparatus 101 stores the generated radiation image data in the memory 111.

To obtain radiation image data at a quality level appropriate for diagnosis, it is necessary to correct the dark noise caused by the radiation imaging apparatus 101. However, in some cases, the user selects and sets the operation conditions of the radiation imaging system depending on the subject, the environments surrounding the subject, or the purpose of the imaging. Further, automatic control such as automatic exposure performed by the radiation imaging system to simplify the user operation may cause a change to the operation conditions. With the changed operation conditions, the characteristics of the radiation imaging apparatus 101 of the radiation imaging system may also be changed. Thus, to obtain correction data under almost the same conditions as those used for capturing an radiation image, it is necessary to obtain the correction data by performing the same imaging operation as that performed on the radiation image at a timing close to when the radiation image has been captured.

Thus, in steps S308 to S311 to be described below, immediately after the radiation image has been captured, the correction data acquisition processing and the correction processing are performed. In parallel with the correction processing, in step S306, the radiation imaging apparatus 101 compresses the information amount of the radiation image data stored in the memory 111, which is not yet corrected, by, for example, thinning the image data using the image compression circuit 107, without waiting for acquiring the correction data. In step S307, the compressed image data is encrypted as first image data by the encryption processing circuit 108 and transferred from the wireless communication circuit 109 to the control PC 301 via the access point 201.

The first image data corresponds to preview image data used for providing an outline of the current imaging result to the operator 312. The purpose of the first image data is to promptly provide the outline of the radiation image data and less emphasis is put on the image quality.

In step S308, in parallel with the transfer of the above-described first image data, the radiation imaging apparatus 101 obtains data of a dark image captured without radiation emission. More precisely, a dark image is captured without radiation emission by the photoelectric conversion element 102, the drive circuit 103, and the A/D conversion circuit 104 that perform the imaging operation capable of reproducing the various parameters measured when the radiation image has been captured by using radiation emission. In step S309, the obtained dark image data is stored in the memory 111. The above-described operation allows obtaining a dark noise component that is substantially the same as that obtained when the radiation image has been captured. The dark image is captured immediately after the radiation image has been captured.

In step S310, the offset correction circuit 106 performs an offset correction on the radiation image data using the dark image data stored in the memory 111. The generated data is second image data. In step S311, the generated second image data is encrypted by the encryption processing circuit 108 and transferred from the wireless communication circuit 109 to the control PC 301 via the access point 201, similarly to the first image data. In the present exemplary embodiment, the dark image is captured after the radiation image is captured. However, an offset correction may be performed on the radiation image data by using the data of a dark image that is captured immediately before the radiation image is captured. In addition, the radiation image data may be further subjected to other correction processing (e.g., gain correction, gradation correction), and the obtained image data may also be used as the second image data.

The first image data is generated for promptly providing an outline of the current imaging result to the user of the radiation imaging system. Thus, the quality of the first image data is not at a satisfactory level for diagnosis. Accordingly, it is necessary to prevent the first image data from being mistakenly used for diagnosis. Under such circumstances, a progress bar is displayed over the first image data on the display 310 to clearly indicate and remind the user that the first image data, which is currently being displayed, is preview image data.

Next, processing for displaying the first image data and the second image data by the control PC 301 will be described in detail. As described above, according to the present exemplary embodiment, while the first image data is being displayed, a progress bar is also displayed as an indicator of progress of the processing until the second image data is displayed on the display 310.

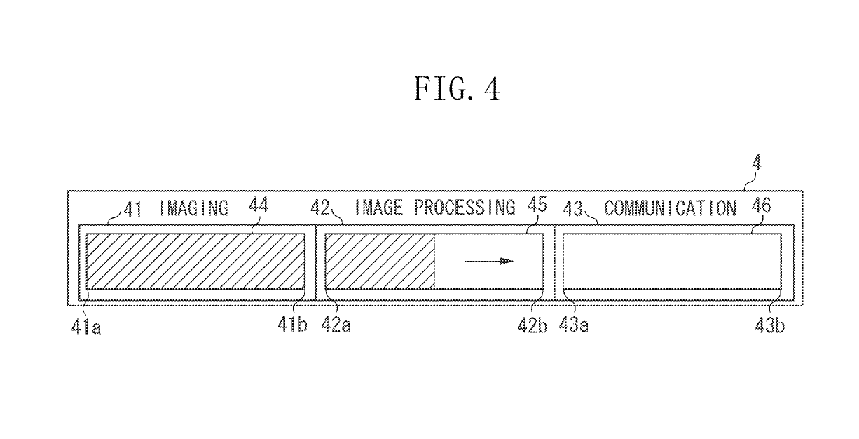

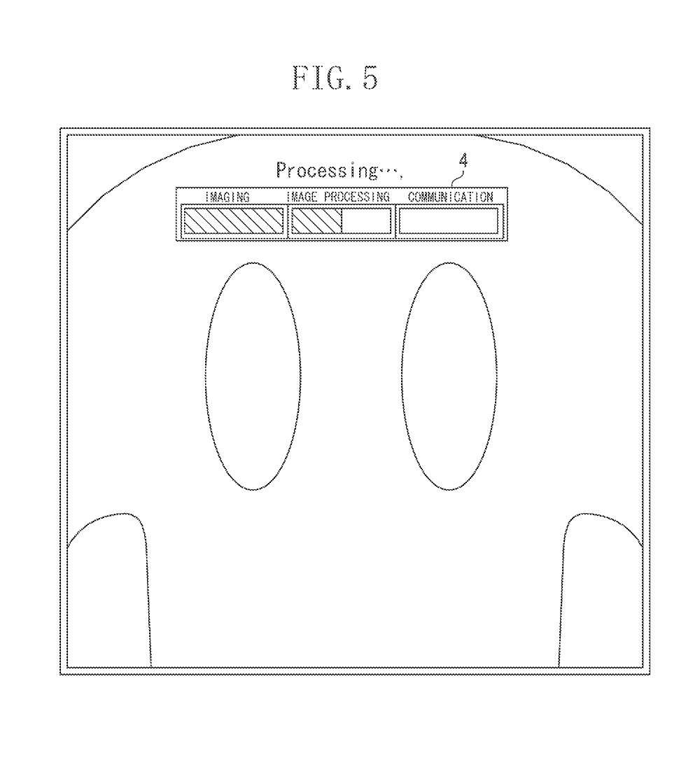

Frame image data 4 illustrated in FIG. 4 is an example of the progress bar displayed on the display 310 by the display management unit 307. The frame image data 4 serving as the progress bar includes frame image data 41, frame image data 42, and frame image data 43. The frame image data 41 indicates the progress of imaging. The frame image data 42 indicates the progress of image processing. The frame image data 43 indicates the progress of communication. Further, in the frame image data 41, the frame image data 42, and the frame image data 43, bar image data 44, bar image data 45, and bar image data 46 are displayed with start points 41a, 42a, and 43a at the left ends thereof and end points 41b, 42b and 43b at the right ends thereof, respectively. In the present exemplary embodiment, as illustrated in FIG. 5, the frame image data 4 is displayed while combined and overlapped with the first image data at a predetermined position.

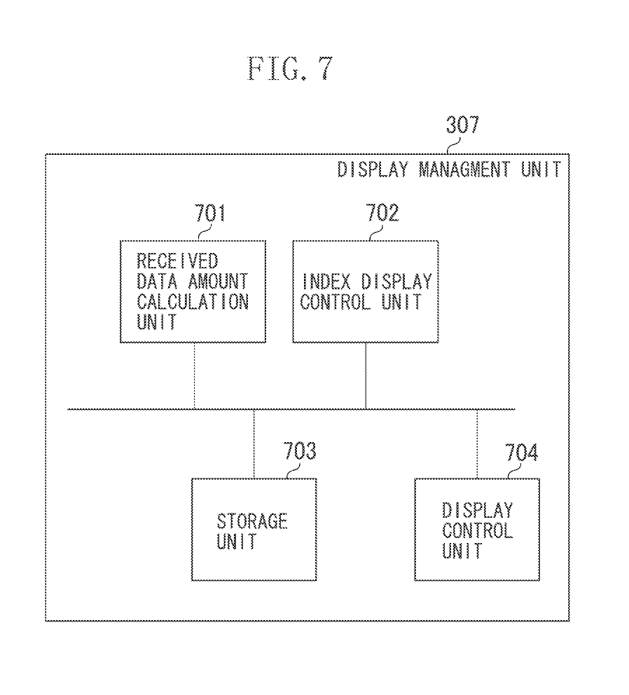

FIG. 7 illustrates a detailed configuration of the display management unit 307 according to the first exemplary embodiment. As illustrated in FIG. 7, the display management unit 307 includes a received data amount calculation unit 701, an index display control unit 702, a storage unit 703, and a display control unit 704. The received data amount calculation unit 701, the index display control unit 702, and the display control unit 704 are functional configurations which are realized when the CPU 313 of the control PC 301 reads out necessary data or program from a recording medium such as a read-only memory (ROM) and executes it. Further, the storage unit 703 is a functional configuration which corresponds to, for example, a part of the storage area of the RAM 306 in the control PC 301.

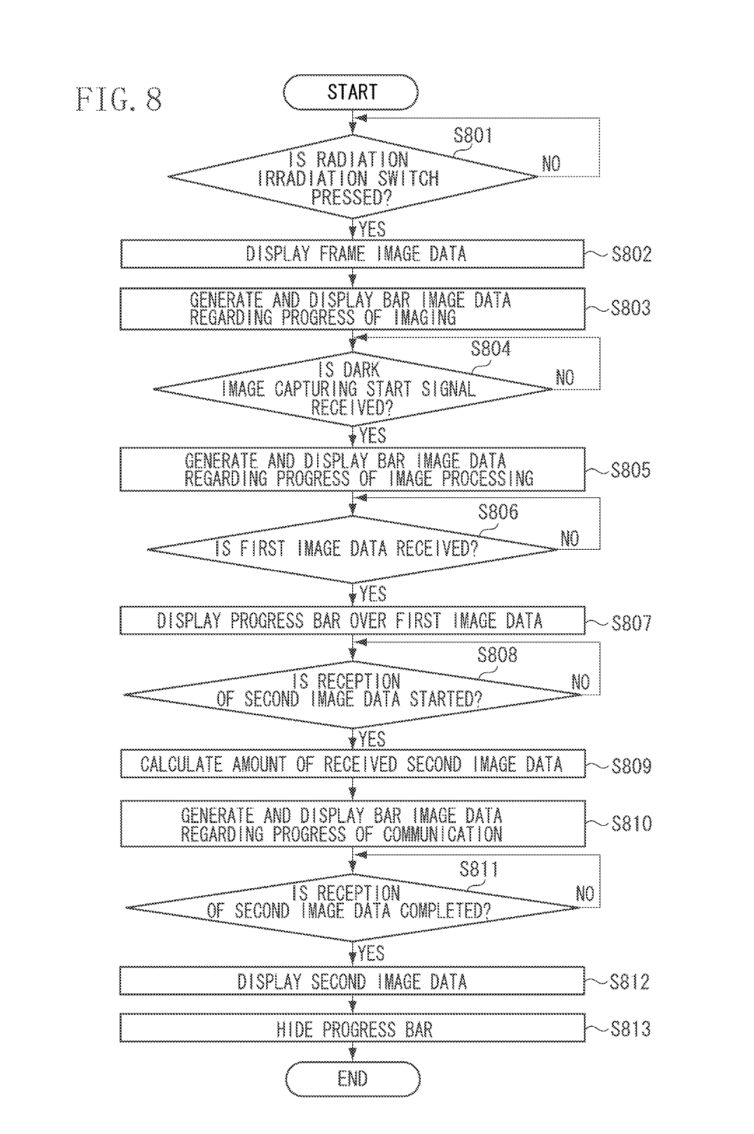

The processing for displaying the first image data and the second image data by the control PC 301 will now be described with reference to the flowchart illustrated in FIG. 8. In step S801, when the user has pressed the radiation irradiation switch 311 (YES in step S801), the processing proceeds to step S802. In step S802, the progress bar, namely the frame image data 4, the frame image data 41 regarding the progress of imaging, the frame image data 42 regarding the progress of image processing, and the frame image data 43 regarding the progress of communication are displayed on the display 310. The coordinate position of the display is stored in advance in the storage unit 703. In step S803, the index display control unit 702 starts generating the bar image data 44 regarding the progress of imaging. The bar image data 44 regarding the progress of imaging is the information that indicates a ratio of time from the reception of an imaging start signal up to the present time to the required imaging time corresponding to the size of radiation image data. The bar image data 44 is displayed at a predetermined coordinate position within the frame of the frame image data 41 regarding the progress of imaging. The required imaging time corresponding to the size of each piece of radiation image data and the above-described predetermined coordinate position are the values previously stored in the storage unit 703.

Thus, on the display 310, the bar image data 44 regarding the progress of imaging, which has the length corresponding to the time from the reception of an imaging start signal up to the present time, is displayed within the frame of the frame image data 41 regarding the progress of imaging. As the time elapses, the bar image data 44 regarding the progress of imaging extends toward the end of the frame.

Subsequently, when the radiation imaging apparatus 101 starts capturing a dark image without radiation emission, a signal indicating the start of image processing is transmitted from the radiation imaging apparatus 101 to the control PC 301 via the wireless communication circuit 109 and the access point 201. In step S804, when the display management unit 307 receives the signal indicating the start of the image processing (YES in step S804), the processing proceeds to step S805. In step S805, the index display control unit 702 starts generating the bar image data 45 regarding the progress of image processing. The bar image data 45 regarding the progress of image processing is the information that indicates a ratio of time from the reception of an image processing start signal up to the present time to the required image processing time corresponding to the size of radiation image data. The bar image data 45 is displayed at a predetermined coordinate position within the frame of the frame image data 42 regarding the progress of image processing. The required image processing time corresponding to the size of each piece of radiation image data and the above-described predetermined coordinate position are the information previously stored in the storage unit 703.

Thus, on the display 310, the bar image data 45 regarding the progress of image processing, which has the length corresponding to the time from the reception of an image processing start signal up to the present time, is displayed within the frame of the frame image data 42 regarding the progress of image processing. As the time elapses, the bar image data 45 regarding the progress of image processing extends toward the end of the frame. In step S806, when the control PC 301 receives the first image data (YES in step S806), the processing proceeds to step S807. In step S807, the index display control unit 702 outputs the received first image data to the display 310 so that the progress bar is displayed over the first image data.

In step S808, when the display management unit 307 starts receiving the second image data (YES in step S808), the processing proceeds to step S809. In step S809, the received data amount calculation unit 701 calculates an amount of the received second image data (hereinafter referred to as a received data amount). For example, when a packet communication method is used, if the received packet includes a fixed amount of image data, the received amount of the second image data up to the present time can be obtained by multiplying the fixed data amount (the number of bits or the number of bytes) by the number of received packets. If the amount of image data in the packet is not fixed, the amount of image data of one packet can be obtained by calculating the amount of data from the beginning of the data to the end-of-data (EOD) code in the packet. The calculation is performed each time a packet is received. Then, the received amount of the second image data up to the present time can be obtained by adding up the data amount of each packet. Further, if data amount information of the image data stored in the packet is included in the header of the packet, by accumulating the data amount information included in each packet each time a packet is received, the received amount of the second image data up to the present time can be obtained. The received amount of the second image data up to the present time is stored in the storage unit 703.

In step S810, the index display control unit 702 obtains a ratio of the received data amount up to the present time to the maximum transmission amount of data to be transmitted from the radiation imaging apparatus 101, and generates the bar image data 46 corresponding to the obtained ratio within the frame of the frame image data 43 regarding the progress of communication. Then, the index display control unit 702 outputs the bar image data 46 to the display 310 together with the coordinate data that indicates the display position of the frame image data 43 regarding the progress of communication. Thus, on the display 310, the bar image data 46 regarding the progress of communication, which has the length corresponding to the received data amount up to the present time, is displayed within the frame of the frame image data 43 regarding the progress of communication. Since the received amount of image data sent from the radiation imaging apparatus 101 increases as the time elapses, the bar image data 46 regarding the progress of communication extends toward the end of the frame.

In step S811, when the index display control unit 702 determines that the received amount of the second image data calculated by the received data amount calculation unit 701 is equal to the maximum transmission amount and the reception of the second image data has been completed (YES in step S811), the processing proceeds to step S812. In step S812, the index display control unit 702 outputs the received second image data to the display 310. Simultaneously, in step S813, the index display control unit 702 disables and hides (terminates) all the displays of the frame image data 4, the frame image data 41 regarding the progress of imaging, the frame image data 42 regarding the progress of image processing, the frame image data 43 regarding the progress of communication, the bar image data 44, the bar image data 45, and the bar image data 46. Accordingly, when the second image data is displayed, only the second image data is displayed on the display 310 without being overlapped with the progress bar.

According to the present exemplary embodiment, although the frame image data 4 is displayed at a predetermined position, the position is not limited thereto. More specifically, the frame image data 4 may be displayed at a position designated by the user by providing the configuration with an input unit for accepting a designation input from the user. Further, according to the present exemplary embodiment, although the frame image data 4 is displayed over the first image data, the frame image data 4 is not necessarily displayed over the first image data. In other words, the frame image data 4 may be displayed at an arbitrary position so long as it is displayed within the display screen of the display 310. For example, the frame image data 4 may be displayed outside the display area of the first image data.

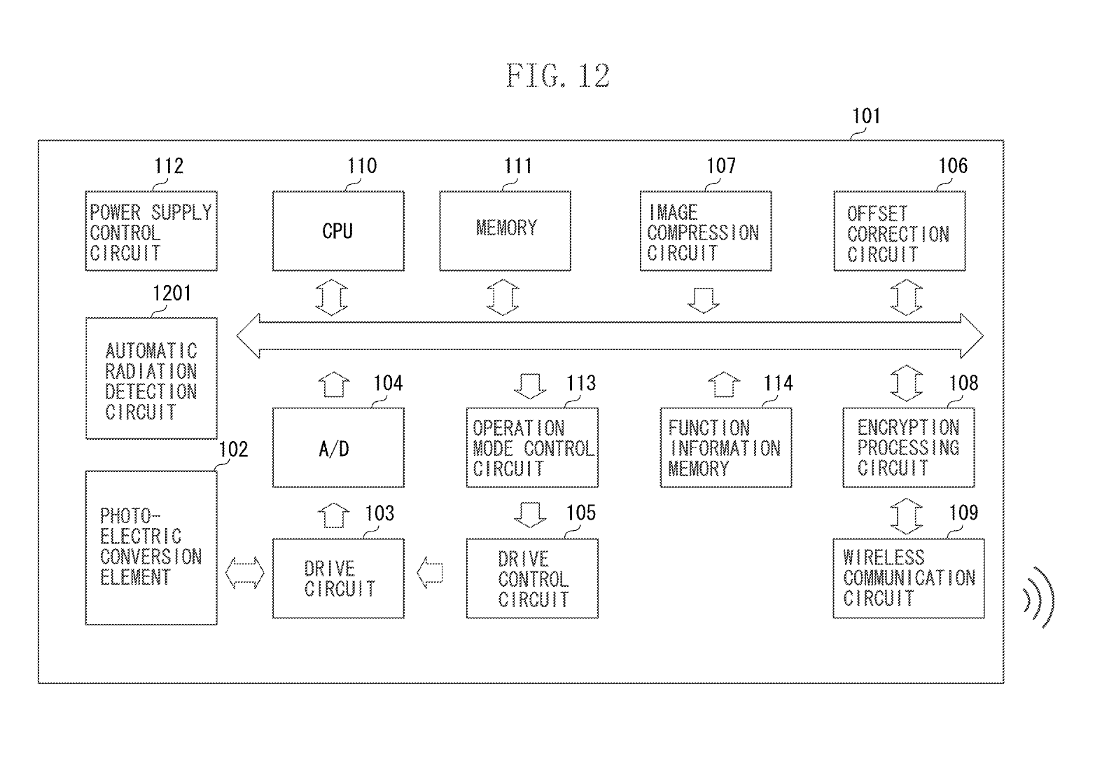

In the present exemplary embodiment, although the operation of the radiation imaging apparatus 101 in the synchronous radiation mode has been described, the imaging may also be performed in the automatic radiation detection mode. FIG. 12 illustrates a block diagram of the radiation imaging apparatus 101 in the automatic radiation detection mode. In FIG. 12, the components of the radiation imaging apparatus 101 which are same as those illustrated in FIG. 2 are denoted by the same reference numerals. Specifically, the radiation imaging apparatus 101 in FIG. 12 is similar to the radiation imaging apparatus 101 in the synchronous radiation mode except that an automatic radiation detection circuit 1201 is added thereto. The automatic radiation detection circuit 1201 monitors the integrated value of the amount of the electric current of the photoelectric conversion element 102 which is arranged two-dimensionally, and detects that incident radiation has been received when the change in the electric current exceeds a predetermined threshold value. Further, whether to display or hide the progress bar may be changed according to the operation mode.

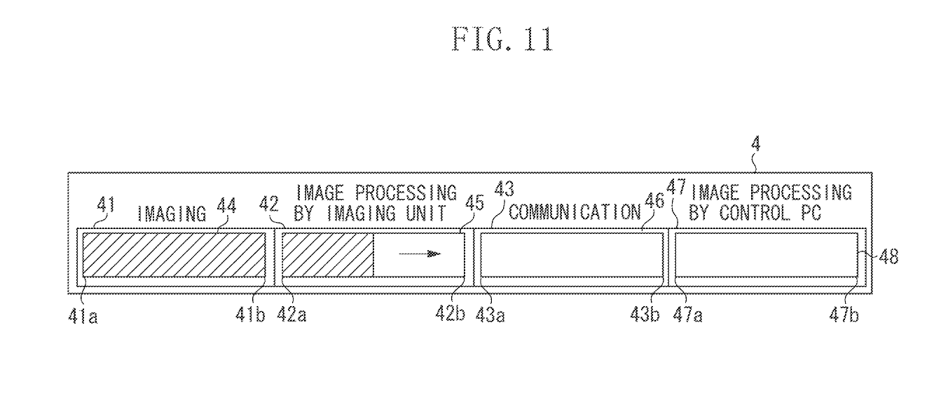

Further, in the present exemplary embodiment, the user is reminded by the display of the progress bar over the first image data that the first image data is preview image data. However, the present exemplary embodiment is not limited thereto. For example, a sand clock or a character string such as “preview image” may be displayed over the first image data. Further, the frame and background color of the first image data may be changed or the brightness of the second image data may be reversed (negative/positive inversion) to clearly show the user that preview image data is being displayed. Furthermore, in the present exemplary embodiment, although image processing such as dark noise correction is performed by the radiation imaging apparatus 101, it may be performed by the control PC 301 or by both the radiation imaging apparatus 101 and the control PC 301. In the present exemplary embodiment, the progress bar separated into three sections (indicating the progress of imaging, the progress of image processing, and the progress of communication) is displayed. However, when image processing is performed by the CPU 313 of the control PC 301 to obtain high-image quality, it takes more time to display the second image data. Thus, as illustrated in FIG. 11, a display indicating that the image processing is being performed by the control PC 301 may also be provided.

As described above, according to the first exemplary embodiment, displaying the first image data and the second image data in different display modes can prevent the first image data from being mistakenly used for diagnosis.

Next, a second exemplary embodiment of the present invention will be described. The components of the radiation imaging system according to the second exemplary embodiment are similar to the components illustrated in FIGS. 1 and 2 according to the first exemplary embodiment except for the display management unit 307. The differences from the first exemplary embodiment will be mainly described below.

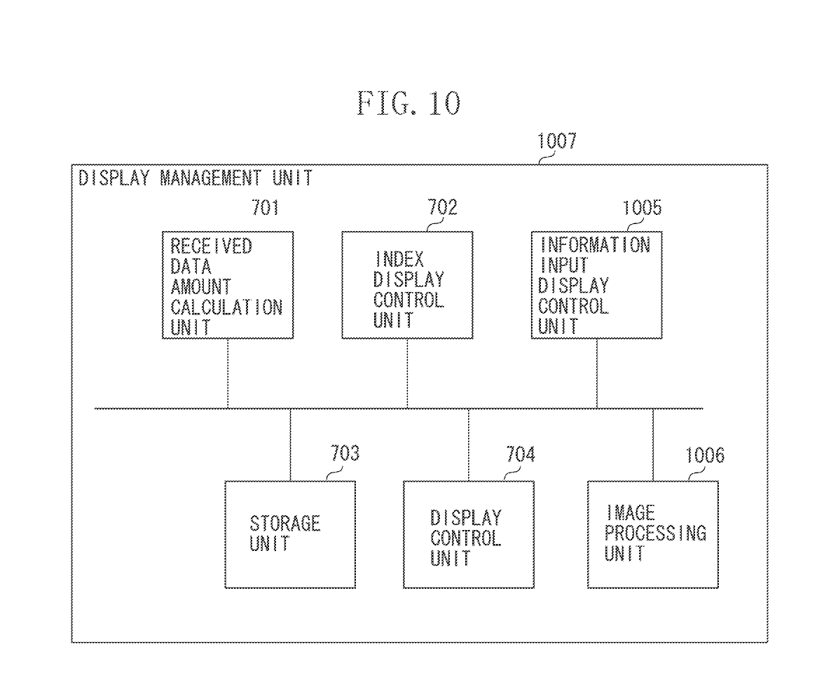

FIG. 10 illustrates a detailed configuration of a display management unit 1007 according to the second exemplary embodiment. In FIG. 10, the components similar to those illustrated in FIG. 7 are denoted by the same reference numerals. Specifically, the display management unit 1007 according to the second exemplary embodiment is similar to the display management unit 307 according to the first exemplary embodiment except that an information input display control unit 1005 and an image processing unit 1006 are added thereto. The information input display control unit 1005 and the image processing unit 1006 are realized by the CPU 313 of the control PC 301 reading out necessary data or program from a recording medium such as a ROM and executing it.

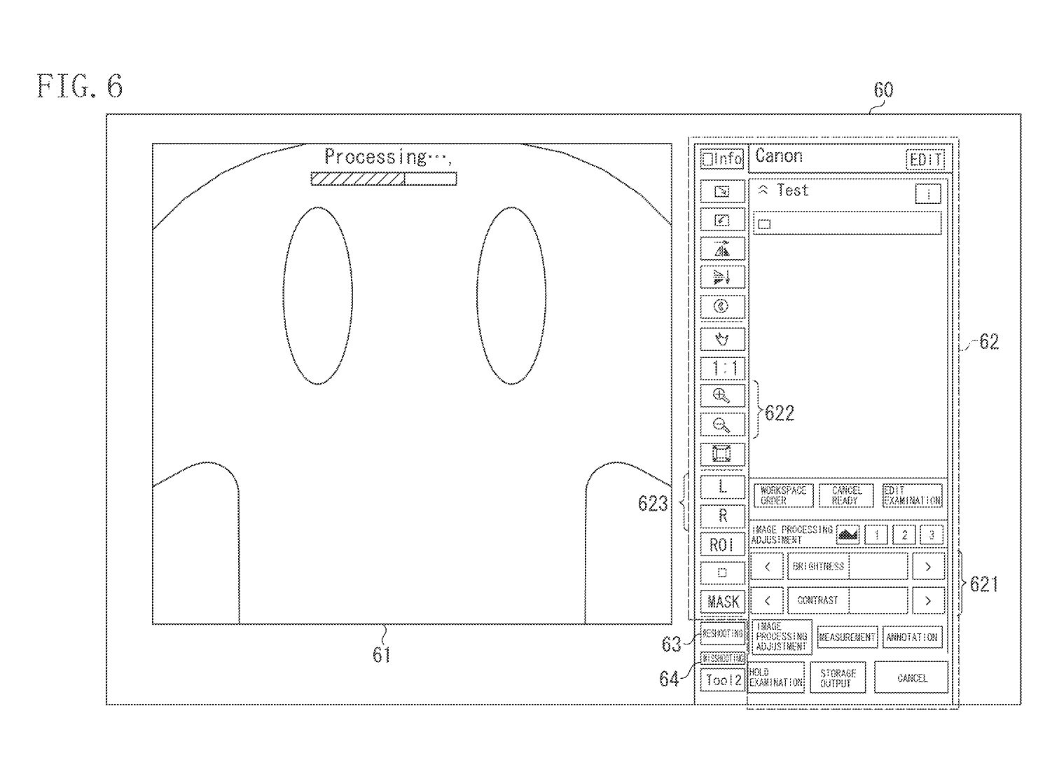

Since the processing for acquiring and displaying the first and second image data according to the second exemplary embodiment is similar to that according to the first exemplary embodiment, the description thereof is omitted. Next, the display screen of the display 310 will be described with reference to FIGS. 10 and 6.

FIG. 6 illustrates an example of a display screen of the display 310. As illustrated in FIG. 6, a display screen 60 of the display 310 includes an image display area 61, various image processing buttons 62, and a reshooting button 63. The image display area 61 is the area where the first and second image data of an image captured by the radiation imaging apparatus 101 is displayed. The progress bar described according to the first exemplary embodiment can also be displayed. The image processing buttons 62 are used when the user performs image processing on the displayed image data. The image processing buttons 62 include, for example, adjustment buttons 621 for adjustment of brightness and contrast, a scaling button 622 for scaling of a designated area, and a flip horizontal button 623. A misshooting button 64 is used to cancel the image processing currently being performed if the misshooting of the displayed captured image is determined after checking the imaging state, for example, whether the exposure is appropriate or whether the image of the body part is captured at an appropriate angle. The reshooting button 63 is used to perform preparations for reshooting by, for example, resetting various parameters for imaging if the misshooting button 64 is pressed to cancel the image processing.

The above-described various parameters are, for example, dose and accumulation time which are parameters of the imaging corresponding to the imaging part as well as various image processing parameters corresponding to the imaging part. When the user presses the radiation irradiation switch 311, an imaging start signal is transmitted to the display control unit 1007. When the display control unit 1007 receives the imaging start signal, the information input display control unit 1005 displays the image processing buttons 62, the reshooting button 63, and the misshooting button 64 in a disabled state.

When the reception of the first image data sent from the wired communication circuit 305 is completed, the display control unit 1007 starts to display the first image data in the image display area 61 of the display 310. In response to this, the information input display control unit 1005 displays the reshooting button 63 and the misshooting button 64 in an enabled state, although the image processing buttons 62 continue to be displayed in a disabled state. If the user presses the reshooting button 63 and the misshooting button 64, the functions of the reshooting processing becomes enabled.

When the user presses the reshooting button 63 and the misshooting button 64, a reshooting signal and various parameters for reshooting stored in the storage unit 703 are transmitted to the radiation imaging apparatus 101 via the wired communication circuit 305, the access point 201, and the wireless communication circuit 109. When the radiation imaging apparatus 101 receives the reshooting signal, the processing regarding the image data currently being processed has been cancelled and the transmitted various parameters are reset by the internal circuit.

When the display control unit 1007 receives the second image data via the wired communication circuit 305, the access point 201, and the wireless communication circuit 109, the display control unit 704 changes the display of the image processing buttons 62 to the enabled state. Further, the display control unit 1007 enables the various images processing functions of the image processing unit 1006.

As described above, the display is controlled so that various types of image processing cannot be performed on the first image data, thereby preventing the first image data from being mistakenly used for diagnosis. Further, if misshooting is determined by checking the first image data, preparations for reshooting the first image can be smoothly started.

The above-described exemplary embodiments can also be realized by supplying a software (program) for implementing the functions of the aforementioned exemplary embodiments to a system or an apparatus via a network or various types of storage media, and causing a computer (or a CPU or a micro processing unit (MPU)) in the system or the apparatus to read and execute the program stored in such storage media.

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

According to the exemplary embodiments of the present invention, it is possible to prevent inappropriate image data to be mistakenly used for diagnosis.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2013-044542 filed Mar. 6, 2013, which is hereby incorporated by reference herein in its entirety.

What is claimed is:

1. A display control apparatus comprising:
a display control unit configured to control a display unit to display first image data and second image data in different display modes on the display unit;
the first image data being generated by detecting, with use of a radiation detection unit, radiation that has transmitted through a subject, and
the second image data being generated by subjecting the first image data to predetermined image processing.

2. The display control apparatus according to claim 1, wherein the display control unit controls the display unit to display predetermined information, which is not displayed when the second image data is displayed, when the first image data is displayed.

3. The display control apparatus according to claim 2, wherein the predetermined information includes at least any one of information associated with imaging processing on the first image data, information associated with the predetermined image processing on the first image data, and information associated with input processing on the second image data.

4. The display control apparatus according to claim 3, wherein at least any one of the information associated with the imaging processing on the first image data and the information associated with the predetermined image processing on the first image data is information previously generated based on a time determined corresponding to the first image data, and the information associated with the input processing on the second image data is information generated based on an input data amount of the second image data.

5. The display control apparatus according to claim 1, wherein the display control unit reverses negative to positive between when the first image data is displayed and when the second image data is displayed.

6. The display control apparatus according to claim 1, wherein the display control unit reverses a background color between when the first image data is displayed and when the second image data is displayed.

7. The display control apparatus according to claim 1, further comprising a control unit configured to prohibit predetermined image processing on the first image data when the first image data is displayed.

8. The display control apparatus according to claim 7, wherein the control unit permits reshooting of the first image when the first image data is displayed.

9. A method for controlling display of image data on a display unit controlled by a display control apparatus, the method comprising:
displaying first image data and second image data in different display modes on the display unit,
the first image data being generated by detecting, with use of a radiation detection unit, radiation that has transmitted through a subject, and
the second image data being generated by subjecting the first image data to predetermined image processing.

10. A non-transitory computer-readable storage medium storing a program that causes a computer to perform a display process, comprising:
displaying first image data and second image data in different display modes on a display unit,
the first image data being generated by detecting, with use of a radiation detection unit, radiation that has transmitted through a subject, and
the second image data being generated by subjecting the first image data to predetermined image processing.

11. An image processing apparatus for receiving an image from a radiation imaging unit that obtains a radiation image by detecting radiation, the image processing apparatus comprising:
a reception unit configured to receive, after receiving a first image of the radiation image data from the radiation imaging unit, a second image of the radiation image data which has a higher resolution than the first image;
an image processing unit configured to process the second image to obtain an image that has been subjected to the image processing; and
a display control unit configured to display a progress display indicating progress of at least any one of processing by the radiation imaging unit and processing by a control apparatus from start of capturing the radiation image to completion of the image processing on the second image, together with the first image on a display unit, and further to terminate, in response to the completion of the image processing on the second image, the display of the progress display and the first image, and to display the second image.

12. The image processing apparatus according to claim 11, wherein the display control unit displays the progress display by overlapping the first image.

13. The image processing apparatus according to claim 11, wherein the display control unit displays on the display unit a progress bar serving as the progress display which changes a display according to a data amount of at least any one of the first image and the second image received by the reception unit.

14. The image processing apparatus according to claim 11, wherein the display control unit displays on the display unit a progress bar serving as the progress display which changes a display according to progress of image processing performed on at least any one of the first image and the second image by the image processing unit.

15. The image processing apparatus according to claim 11, wherein the reception unit receives the first image which is image data not subjected to difference processing using dark current data obtained by the radiation imaging unit, and the second image which is image data subjected to the difference processing using the dark current data obtained by the radiation imaging unit.

16. A radiation imaging system comprising:
an image processing apparatus according to claim 11;
a radiation imaging unit, including a connector connectable to a wireless communication unit and a wired communication unit, configured to transfer a radiation image to the image processing apparatus via the wireless communication unit and the connector.

17. A method for processing an image, the method comprising:
receiving a first image of radiation image data from a radiation imaging unit;
receiving a second image of the radiation image data which has a higher resolution than the first image;
performing image processing on the received second image and obtaining an image that has been subjected to the image processing;
displaying a progress display indicating progress of at least any one of processing by the radiation imaging unit and processing by a control apparatus from start of capturing the radiation image to completion of the image processing on the second image, together with the first image on a display unit;
terminating, in response to the completion of the image processing on the second image, the display of the progress display and the first image; and
displaying, in response to the completion of the image processing on the second image, the image obtained by the image processing.

18. A non-transitory computer-readable storage medium storing a program that causes a computer to execute the method according to claim 17.

19. A display control apparatus comprising:
an obtaining unit configured to obtain first image data generated by a radiation detection unit for detecting radiation passed through a subject, and second image data generated by subjecting the obtained first image data to predetermined image processing; and
a display control unit configured to cause a display unit to display the first image data together with information indicating that a predetermined process is being performed, and to terminate display of the information and display the second image data instead of the first image data, without displaying the information.

20. A display control method comprising:
obtaining first image data generated by a radiation detection unit for detecting radiation passed through a subject;
obtaining second image data generated by subjecting the obtained first image data to predetermined image processing;
causing a display unit to display the first image data together with information indicating that a predetermined process is being performed;
terminate display of the information; and
displaying the second image data instead of the first image data, without displaying the information.
